(19) United States
(12) Patent Application Publication
(10) Pub. No.: US 2023/0368421 A1
(43) Pub. Date: Nov. 16, 2023

(54) RADIATION THERAPY DEVICE, MEDICAL IMAGE PROCESSING DEVICE, RADIATION THERAPY METHOD, AND STORAGE MEDIUM

(71) Applicants: Toshiba Energy Systems & Solutions Corporation, Kawasaki-shi (JP); National Institutes for Quantum Science and Technology, Chiba-shi (JP)

(72) Inventors: Yukinobu SAKATA, Kawasaki Kanagawa (JP); Kenta UMENE, Fuchu Tokyo (JP); Ryusuke HIRAI, Meguro Tokyo (JP); Akiyuki TANIZAWA, Kawasaki Kanagawa (JP); Shinichiro MORI, Chiba-shi (JP); Keiko OKAYA, Setagaya Tokyo (JP)

(73) Assignees: Toshiba Energy Systems & Solutions Corporation, Kawasaki-shi (JP); National Institutes for Quantum Science and Technology, Chiba-shi (JP)

(21) Appl. No.: 18/351,276

(22) Filed: Jul. 12, 2023

Related U.S. Application Data
(63) Continuation of application No. PCT/JP22/07513, filed on Feb. 24, 2022.

(30) Foreign Application Priority Data
Feb. 26, 2021 (JP) 2021-030869

(51) Int. Cl. G06T 7/73; G06T 15/00; A61N 5/10; G01T 1/17

(52) U.S. Cl. CPC G06T 7/74; G06T 15/00; A61N 5/1049; G01T 1/17; G06T 2200/04; G06T 2207/10121; G06T 2207/20212; G06T 2207/30004; G06T 2210/41; G06T 2207/10081; A61N 2005/1062

(57) ABSTRACT

According to an embodiment, a radiation therapy device includes an acquirer, a projection position calculator, an element projection image generator, and an element projection image synthesizer. The acquirer acquires a condition of X-ray imaging in a treatment stage and a three-dimensional image of a patient imaged before the treatment stage. The projection position calculator calculates a projection position when each of pixels included in the three-dimensional image is projected onto a two-dimensional X-ray fluoroscopic image generated in the X-ray imaging on the basis of the condition of the X-ray imaging. The element projection image generator generates an element projection image for each pixel when each of the pixels included in the three-dimensional image is projected onto the X-ray fluoroscopic image. The element projection image synthesizer performs a synthesis process for the element projection image for each pixel on the basis of the projection position to generate a reconstructed image.

### CROSS-REFERENCE TO RELATED APPLICATION

This application is based upon and claims the benefit of priority from Japanese Patent Application No. 2021-030869, filed Feb. 26, 2021 and PCT/JP2022/007513, filed Feb. 24, 2022; the entire contents all of which are incorporated herein by reference.

### FIELD

Embodiments of the present invention relate to a radiation therapy device, a medical image processing device, a radiation therapy method, and a storage medium.

### BACKGROUND

Radiation therapy is a treatment method of irradiating a diseased part within a patient's body with radiation to destroy the diseased part. In the radiation therapy, the radiation is required to be accurately radiated to a position of the diseased part so that normal tissue is not damaged. Thus, before radiation irradiation starts, the position of the diseased part is identified using an X-ray fluoroscopic image or the like, a position and angle of a movable treatment bed on which the patient is placed are adjusted appropriately, and the diseased part is accurately aligned in a radiation irradiation range. Such alignment is performed by collating a digitally reconstructed radiograph (DRR) obtained by virtually reconstructing an X-ray fluoroscopic image from a three-dimensional computed tomography (CT) image obtained by performing CT in advance in a treatment planning stage with an X-ray fluoroscopic image captured in a treatment stage.

In the above-described alignment, a movement amount of the patient is obtained by solving a six-dimensional (three-dimensional translation and three-dimensional rotation) search problem using a degree of similarity between the X-ray fluoroscopic image captured in the treatment stage and the DRR as an index. Because it is difficult to analytically solve this search problem, the search problem is generally solved through repeated calculations and it takes a long time to achieve high-precision alignment. In particular, because an amount of calculation required for DRR generation is large and a calculation process occupies most of the processing time, it is necessary to reduce the number of times the DRR is generated or increase the generation speed in order to implement high-speed alignment.

In order to shorten the processing time, a method of reducing the number of times the DRR is generated and achieving high-speed alignment by evaluating the degree of similarity between the DRR and the X-ray fluoroscopic image only in one direction in which there is a significant change in the image has been proposed. In this conventional method, it is possible to reduce the number of times the DRR is generated. However, because a ray tracing method, which requires a large amount of calculation and a long processing time, is used for DRR generation, it is difficult to shorten a period of time required for one DRR generation process and it is still difficult to implement high-speed alignment.

### BRIEF DESCRIPTION OF DRAWINGS

FIG. 1 is a block diagram showing a schematic configuration of a radiation therapy system including a radiation therapy device according to an embodiment.

FIG. 2 is a diagram for describing a projection matrix for use in a projection position calculation process according to the embodiment.

FIG. 3A is a diagram showing a state in which a DRR is generated in a conventional ray tracing method.

FIG. 3B is a diagram showing a state in which a DRR is generated by a DRR generator according to the embodiment.

FIG. 4 is a functional block diagram showing a schematic configuration of a DRR generator according to the embodiment.

FIG. 5 is a flowchart showing an example of a processing flow of the radiation therapy system according to the embodiment.

FIG. 6 is a flowchart showing an example of a flow of a DRR generation process of the DRR generator according to the embodiment.

FIG. 7 is a diagram showing a state in which an element projection image is generated by the DRR generator according to the embodiment.

FIG. 8 is an image diagram of a DRR generated by the DRR generator according to the embodiment.

FIG. 9 is a diagram showing experimental results of positioning processes of the radiation therapy device according to the embodiment and a device of a comparative example.

### DETAILED DESCRIPTION

A radiation therapy device, a medical image processing device, a radiation therapy method, and a storage medium according to embodiments will be described below with reference to the drawings.

According to an embodiment, a radiation therapy device includes an acquirer, a projection position calculator, an element projection image generator, and an element projection image synthesizer. The acquirer acquires a condition of X-ray imaging in a treatment stage and a three-dimensional image of a patient imaged before the treatment stage. The projection position calculator calculates a projection position when each of pixels included in the three-dimensional image is projected onto a two-dimensional X-ray fluoroscopic image generated in the X-ray imaging on the basis of the condition of the X-ray imaging. The element projection image generator generates an element projection image for each pixel when each of the pixels included in the three-dimensional image is projected onto the X-ray fluoroscopic image. The element projection image synthesizer performs a synthesis process for the generated element projection image for each pixel on the basis of the calculated projection position to generate a reconstructed image virtually reproducing the X-ray fluoroscopic image from the three-dimensional image.

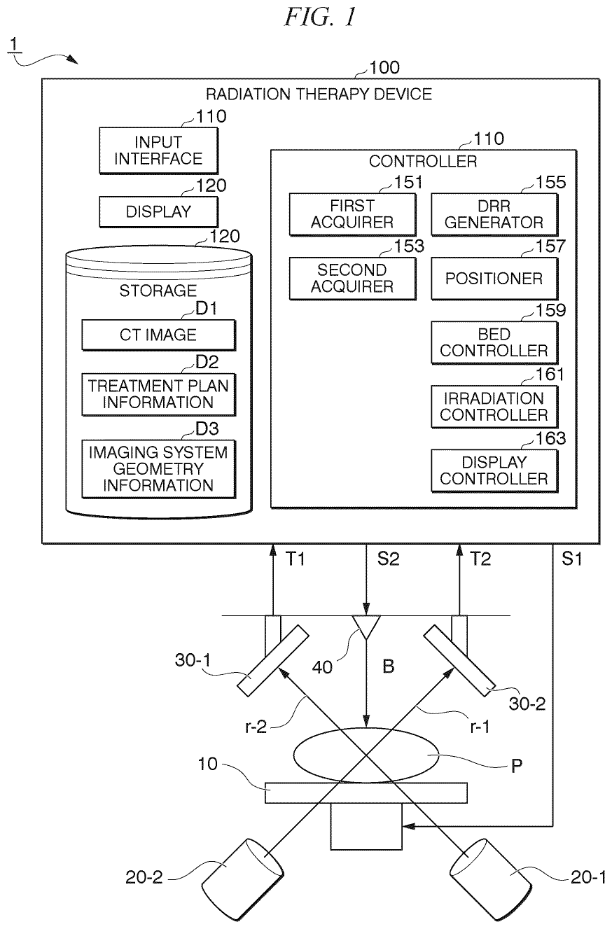

FIG. 1 is a block diagram showing a schematic configuration of a radiation therapy system including the radiation therapy device according to an embodiment. The radiation therapy system 1 includes, for example, a treatment bed 10, two radiation sources 20 (a radiation source 20-1 and a radiation source 20-2), two radiation detectors 30 (a radiation detector 30-1 and a radiation detector 30-2), a treatment beam irradiation gate 40, and a radiation therapy device 100. The radiation therapy device 100 is an example of a “radiation therapy device” or a “medical image processing device.”

The treatment bed 10 is a bed on which a subject (patient) P to be treated with radiation is placed and fixed. The treatment bed 10 includes a translation mechanism and a rotation mechanism for changing a direction of a treatment beam with which the fixed patient P is irradiated. The treatment bed 10 can be moved in three axial directions or six axial directions by the translation mechanism and the rotation mechanism.

The radiation source 20-1 radiates radiation r-1 for seeing through the body of the patient P at a predetermined angle. The radiation source 20-2 radiates radiation r-2 for seeing through the body of the patient P at a predetermined angle different from that of the radiation source 20-1. The radiation r-1 and the radiation r-2 are, for example, X-rays. In FIG. 1, a case in which X-ray photography is performed from two directions on the patient P fixed on the treatment bed 10 is shown. Also, the illustration of a controller that controls the irradiation with the radiation r by the radiation source 20 is omitted from FIG. 1.

The radiation detector 30-1 detects the radiation r-1 which has been radiated from the radiation source 20-1 and has arrived at the radiation detector 30-1 after passing through the inside of the body of the patient P and generates an X-ray fluoroscopic image of the inside of the body of the patient P according to a magnitude of energy of the detected radiation r-1. The radiation detector 30-2 detects the radiation r-2 which has been radiated from the radiation source 20-2 and has arrived at the radiation detector 30-2 after passing through the inside of the body of the patient P and generates an X-ray fluoroscopic image of the inside of the body of the patient P according to a magnitude of energy of the detected radiation r-2.

The radiation detectors 30 include X-ray detectors arranged in a two-dimensional array shape. The radiation detector 30 generates a digital image in which a magnitude of energy of the radiation r arriving at each X-ray detector is represented by a digital value as an X-ray fluoroscopic image. The radiation detector 30 is, for example, a flat panel detector (FPD). The radiation detectors 30-1 and 30-2 output generated X-ray fluoroscopic images T1 and T2 to the radiation therapy device 100. Also, the illustration of a controller that controls the generation of the X-ray fluoroscopic image by the radiation detector 30 is omitted from FIG. 1.

In the radiation therapy system 1, because the positions of the radiation source 20 and the radiation detector 30 are fixed, a direction in which an imaging device including a set of the radiation source 20 and the radiation detector 30 captures an image (a relative direction for a fixed coordinate system of a treatment room) is fixed. Thus, when three-dimensional coordinates are defined in the three-dimensional space where the radiation therapy system 1 is installed, the positions of the radiation source 20 and the radiation detector 30 can be represented by three-axis coordinate values. In the following description, information about the three-axis coordinate values will be referred to as imaging system geometry information of an imaging device including a set of the radiation source 20 and the radiation detector 30. The imaging system geometry information includes information such as a position of the radiation source 20 and a position and tilt of the radiation detector 30. Using the imaging system geometry information, the position of the patient P within prescribed three-dimensional coordinates can be obtained from a position of the time when the radiation radiated from the radiation source 20 passes through the body of the patient P and reaches the radiation detector 30.

The imaging system geometry information can be obtained from installation positions of the radiation source 20 and the radiation detector 30 designed when the radiation therapy system 1 is installed. Alternatively, geometry information can also be obtained from the installation positions of the radiation source 20 and the radiation detector 30 measured by a three-dimensional measurement instrument or the like. By obtaining a projection matrix from the imaging system geometry information, the radiation therapy device 100 can calculate a position (a projection position) of a captured two-dimensional fluoroscopic image in which the patient P within the three-dimensional space has been imaged (a position onto which each point within the three-dimensional space is projected on the DRR).

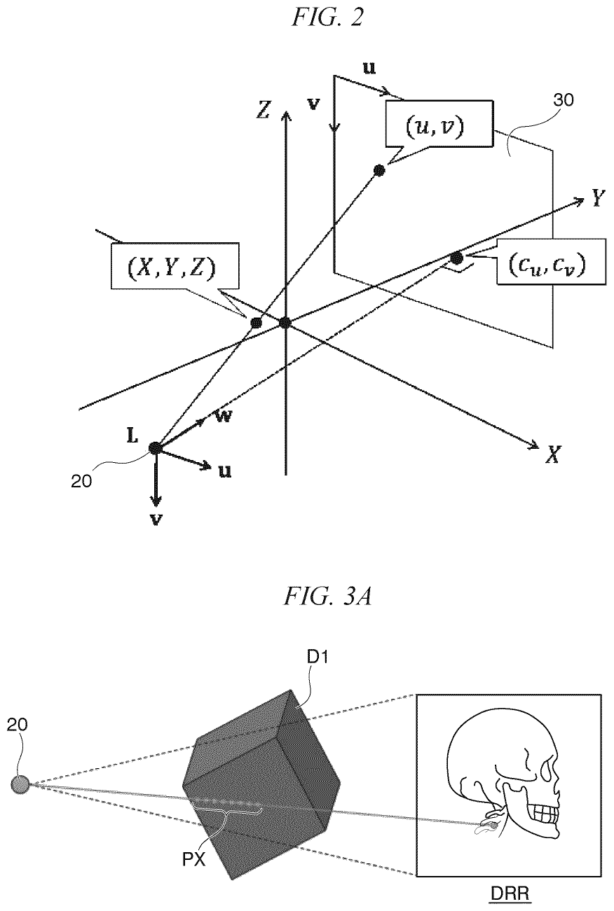

FIG. 2 is a diagram for describing a projection matrix for use in a projection position calculation process according to the embodiment. A projection matrix P is a matrix representing a corresponding relationship when a point within the three-dimensional space is projected onto a two-dimensional fluoroscopic image. A relationship between a point X(→)=(X, Y, Z)^{t} (where (→) denotes a vector) within the three-dimensional space and a point u=(u, v)^{t} on the two-dimensional fluoroscopic image of a projection destination is represented by the following Eq. (1).

$$\begin{matrix}[ Math . 1 ] & \\ λ [ \begin{matrix}u \\ 1\end{matrix} ] = P [ \begin{matrix}X \\ 1\end{matrix} ] & ( 1 )\end{matrix}$$

The projection matrix P is represented by the following Eqs. (2) and (3). In Eqs. (2) and (3), a position of the radiation source 20 is denoted by L(→)=(l_{X}, l_{Y}, l_{Z})^{t}, basis vectors of the radiation detector 30 (FPD) are denoted by u(→)=(u_{X}, u_{Y}, u)^{t}, v(→)=(v_{X}, v, v_{Z})^{t}, and w(→)=(w_{X}, w_{Y}, w)^{t}, a point onto which L(→) is projected on the radiation detector 30 is denoted by c(→)=(cᵤ, cᵥ)^{t}, a distance from L(→) to c(→) is denoted by f, and the pixel pitches of the radiation detectors 30 are denoted by sᵤ [mm/pixel] and sᵥ [mm/pixel].

$$\begin{matrix}[ Math . 2 ] & \\ P = [ \begin{matrix}\frac{f}{{s}_{u}} & 0 & {c}_{u} \\ 0 & \frac{f}{{s}_{v}} & {c}_{v} \\ 0 & 0 & 1\end{matrix} ] [ {R}^{t} - {R}^{t} L ] & ( 2 )\end{matrix}$$

$$\begin{matrix}[ Math . 3 ] & \\ R = [ uvw ] & ( 3 )\end{matrix}$$

Also, in an imaging device that simultaneously captures two fluoroscopic images of the patient P as shown in FIG. 1, a projection matrix is obtained for each set of the radiation source 20 and the radiation detector 30. Thereby, it is possible to calculate a coordinate value of prescribed three-dimensional coordinates indicating a position of a diseased part or a marker from the position of the diseased part such as a lesion or a bone in the body of the patient P imaged in two fluoroscopic images or an image of the marker placed in advance in the body of the patient P.

Also, in FIG. 1, the configuration of the radiation therapy system 1 including two sets of radiation sources 20 and radiation detectors 30, i.e., two imaging devices, is shown. The radiation therapy system 1 may include three or more imaging devices (three or more sets of radiation sources 20 and radiation detectors 30). Also, the radiation therapy system 1 may include only one imaging device (one set of the radiation source 20 and the radiation detector 30).

The treatment beam irradiation gate 40 radiates radiation for destroying a diseased part, which is a treatment target part in the patient P's body, as a treatment beam B. The treatment beam B is, for example, X-rays, y-rays, an electron beam, a proton beam, a neutron beam, a heavy particle beam, or the like. The treatment beam B is linearly radiated to the patient P from the treatment beam irradiation gate 40. Although a configuration of the radiation therapy system 1 including one fixed treatment beam irradiation gate 40 is shown in FIG. 1, the present invention is not limited thereto. The radiation therapy system 1 may include a plurality of treatment beam irradiation gates.

The radiation therapy device 100 controls operations of functions of the radiation therapy system 1. The radiation therapy device 100 includes, for example, an input interface 110, a display 120, a storage 130, and a controller 140. Also, these functional units may be provided in a plurality of devices in a distributed manner. For example, a DRR generation function of the controller 140 may be implemented by a processing device separate from the radiation therapy device 100. This processing device is an example of a “medical image processing device.”

The input interface 110 receives various types of input operations from a radiation therapy practitioner (a doctor, a technician, or the like) who uses the radiation therapy system 1 and outputs a signal indicating the received input operation to the controller 140. The input interface 110 is, for example, a keyboard, a mouse, a touch panel, or the like.

The display 120 displays information such as a CT image, a DRR, an X-ray fluoroscopic image, a current position of the patient P, and a predetermined suitable position for performing radiation therapy (hereinafter referred to as a “preferred position”). The display 120 is, for example, a liquid crystal display (LCD). When the input interface 110 is implemented by a touch panel, the functions of the display 120 may be incorporated into the touch panel.

The storage 130 stores various types of information necessary for radiation therapy. For example, the storage 130 stores a three-dimensional image that enables a process of seeing through the inside of the body of the patient P imaged in the treatment planning stage. The three-dimensional image is, for example, three-dimensional image data acquired by imaging the patient P with an imaging device such as a CT device, a cone-beam (CB) CT device, or a magnetic resonance imaging (MRI) device. In the following description, a case where the three-dimensional image is a CT image D1 obtained by imaging the patient P with the CT device will be described as an example. In addition, the storage 130 stores, for example, treatment plan information D2 such as an irradiation position, an irradiation direction, an irradiation level, and the number of times the radiation beam B is radiated for each patient decided in the treatment planning stage, imaging system geometry information D3, and the like. The storage 130 is implemented by, for example, a random-access memory (RAM), a read-only memory (ROM), a hard disk drive (HDD), and the like.

The controller 140 controls operations for implementing various types of functions of the radiation therapy system 1. The controller 140 includes, for example, a first acquirer 151, a second acquirer 153, a DRR generator 155, a positioner 157, a bed controller 159, an irradiation controller 161, and a display controller 163.

The first acquirer 151 acquires the CT image D1 of the patient P, the treatment plan information D2 of the patient P, and the imaging system geometry information D3 from the storage 130. Also, the first acquirer 151 may acquire the CT image D1 or the like on the basis of information input via the input interface 110. Also, the first acquirer 151 may acquire the CT image D1 or the like from a database (a file server or the like) connected via a network. Also, the first acquirer 151 may acquire the CT image D1 from a storage medium such as a DVD or CD-ROM via a drive device attached to the radiation therapy device 100. That is, the first acquirer 151 acquires a condition of X-ray imaging in the treatment stage and a three-dimensional image of the patient captured before the treatment stage. The first acquirer 151 is an example of an “acquirer.”

The second acquirer 153 acquires X-ray fluoroscopic images T1 and T2 input from the radiation detectors 30-1 and 30-2 in the treatment stage.

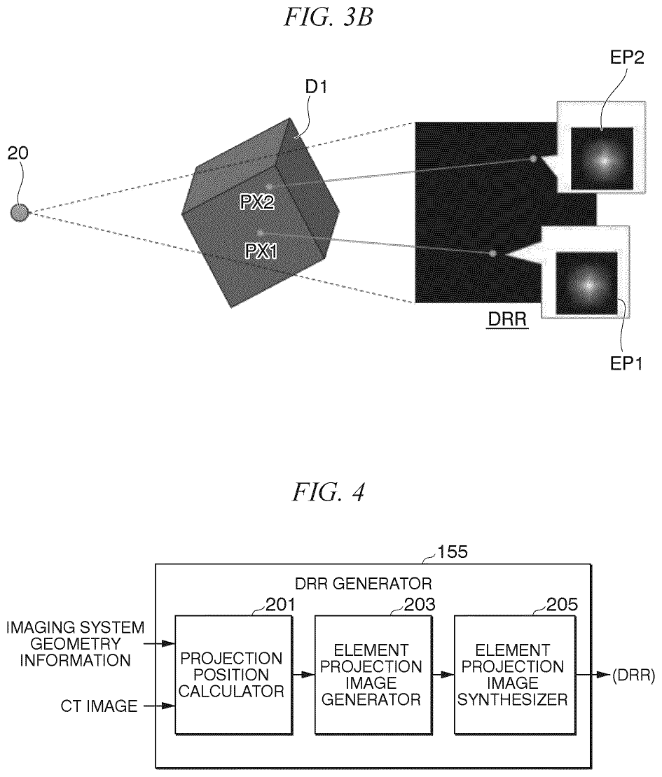

The DRR generator 155 generates a DRR on the basis of the CT image D1 and the imaging system geometry information D3 acquired by the first acquirer 151. FIG. 3A is a diagram showing a state in which a DRR is generated in a conventional ray tracing method. FIG. 3B is a diagram showing a state in which a DRR is generated by the DRR generator 155 according to the embodiment.

As shown in FIG. 3A, in the conventional ray tracing method, the CT image D1 is virtually arranged between the radiation source 20 and the DRR. A luminance value of each pixel of the DRR is obtained by performing an integral process for the luminance value of each pixel PX of the CT image D1 on an X-ray path connecting the radiation source 20 and the pixel. In this case, the X-ray path is sampled at short intervals and the luminance values of the CT image D1 are added. That is, it is necessary to refer to the luminance of the pixel of the CT image D1 through which the X-ray passes and perform an integral process for each pixel of the DRR and an amount of calculation is large. Although a high-precision DRR can be generated by shortening the sampling interval and increasing the number of times of sampling, there is a tradeoff between the quality of the DRR and the processing time because the processing time increases as the number of times of sampling increases. A sampling interval equal to or less than the pixel pitch of the CT image D1 is desirable to generate a DRR with sufficient image quality for positioning.

On the other hand, as shown in FIG. 3B, in the DRR generation process of the DRR generator 155, a DRR is generated on the basis of information about the projection position of the DRR onto which each pixel of the CT image D1 is projected and information of projected pixels (hereinafter referred to as “element projection images”) instead of using the information of the X-ray path. Because the DRR is obtained by projecting the CT image D1, the DRR generator 155 can generate the DRR by superimposing element projection images of all pixels of the CT image D1. For example, in the example shown in FIG. 3B, the DRR generator 155 generates an element projection image EP1 corresponding to a representative pixel PX1 (hereinafter referred to as a “reference pixel”) in the CT image D1 and generates an element projection image (an element projection image EP2 or the like) corresponding to another pixel by two-dimensionally converting the element projection image EP1 that has been generated. Also, when a DRR is generated from these element projection images, the amount of calculation depends only on the number of pixels of the CT image D1, unlike the case of using ray tracing. Thus, the processing time for generating the DRR can be shortened.

FIG. 4 is a functional block diagram showing a schematic configuration of the DRR generator 155 according to the embodiment. The DRR generator 155 includes, for example, a projection position calculator 201, an element projection image generator 203, and an element projection image synthesizer 205.

The projection position calculator 201 calculates a projection position when each pixel of the CT image D1 is projected onto the DRR on the basis of the imaging system geometry information D3. Information such as a three-dimensional position and a rotation angle is set in the CT image D1 on the basis of a treatment plan. The projection position calculator 201 converts a three-dimensional image coordinate system x(→)=(x, y, z)^{t} set in the CT image D1 into a room coordinate system X(→)=(X, Y, Z)^{t} according to the following Eq. (4). A position where one point in the room coordinate system is projected on the DRR can be calculated on the basis of the imaging system geometry information D3. In Eq. (4), A denotes a prescribed transformation matrix set on the basis of the imaging system geometry information D3. Furthermore, the projection position calculator 201 calculates a DRR coordinate system u(→)=(u, v)^{t} from the room coordinate system X(→) according to the following Eq. (5). In Eq. (5), P denotes a projection matrix.

$$\begin{matrix}[ Math . 4 ] & \\ X = A [ \begin{matrix}x \\ 1\end{matrix} ] & ( 4 )\end{matrix}$$

$$\begin{matrix}[ Math . 5 ] & \\ λ [ \begin{matrix}u \\ 1\end{matrix} ] = P [ \begin{matrix}X \\ 1\end{matrix} ] & ( 5 )\end{matrix}$$

That is, the projection position calculator 201 calculates a projection position when each of the pixels included in the three-dimensional image is projected onto a two-dimensional X-ray fluoroscopic image generated in X-ray imaging on the basis of a condition of the X-ray imaging.

The element projection image generator 203 generates an element projection image when each pixel of the CT image D1 is projected onto the DRR. However, it takes a long time to generate accurate element projection images for all pixels included in the CT image D1. Thus, the element projection image generator 203 first generates an element projection image for a reference pixel and two-dimensionally converts the generated element projection image to approximately generate an element projection image for another pixel. That is, the element projection image generator 203 generates an element projection image for each pixel when each pixel included in the three-dimensional image is projected onto the X-ray fluoroscopic image. The element projection image generator 203 generates an element projection image of a reference pixel included in a three-dimensional image and performs a two-dimensional conversion process for the generated element projection image of the reference pixel to generate an element projection image of a pixel other than the reference image included in the three-dimensional image.

The element projection image synthesizer 205 generates a DRR by attaching the element projection images generated by the element projection image generator 203 to projection positions and synthesizing the element projection images. Basically, because the size of an element projection image is one or more pixels and a plurality of element projection images are superimposed on each pixel of the DRR, the element projection image synthesizer 205 adds luminance values during synthesis. That is, the element projection image synthesizer 205 generates a reconstructed image obtained by virtually reproducing an X-ray fluoroscopic image from the three-dimensional image by performing a synthesis process for the element projection image generated for each pixel on the basis of the calculated projection position. Details of the processes of the projection position calculator 201, the element projection image generator 203, and the element projection image synthesizer 205 will be described below.

Returning to FIG. 1, the positioner 157 collates the DRR generated by the DRR generator 155 with the X-ray fluoroscopic images T1 and T2 acquired by the second acquirer 153, and decides a position of the patient P suitable for performing the radiation therapy. Also, the positioner 157 calculates an amount of movement of the treatment bed 10 for moving the current position of the patient P fixed on the treatment bed 10 to the position suitable for performing the radiation therapy. In other words, the positioner 157 calculates an amount of movement of the treatment bed 10 necessary to irradiate a treatment area with the treatment beam B from the irradiation direction predetermined for the CT image D1 in the planning stage at the current position of the patient P. The positioner 157 outputs the calculated amount of movement to the bed controller 159. That is, the positioner 157 positions the patient on the basis of the generated reconstructed image.

The bed controller 159 controls the translation mechanism and the rotation mechanism provided on the treatment bed 10 to change the position and direction of the patient P fixed on the treatment bed 10 on the basis of information about the amount of movement output by the positioner 157. The bed controller 159 outputs a signal S1 indicating the amount of movement to the treatment bed 10. The bed controller 159 controls, for example, the translation mechanism and the rotation mechanism of the treatment bed 10 in three axial directions or six axial directions.

The irradiation controller 161 controls irradiation of the treatment beam B by the treatment beam irradiation gate 40. The irradiation controller 161 outputs a signal S2 indicating an irradiation timing of the treatment beam B to the treatment beam irradiation gate 40 on the basis of the treatment plan information D2 acquired by the first acquirer 151 and the X-ray fluoroscopic images T1 and T2 acquired in real-time in the treatment stage by the second acquirer 153.

The display controller 163 controls the display 120 to display a CT image, a DRR, an X-ray fluoroscopic image, and information such as a current position and a suitable position of the patient P.

For example, a hardware processor such as a central processing unit (CPU) and a storage device (a storage device including a non-transitory storage medium) storing a program (software) are provided for some or all functions of the controller 140 of the above-described radiation therapy device 100 and various types of functions may be implemented by the processor executing the program. Also, some or all functions of the controller 140 of the above-described radiation therapy device 100 may be implemented by hardware (including a circuit unit; circuitry) such as a large-scale integration (LSI) circuit, an application-specific integrated circuit (ASIC), a field-programmable gate array (FPGA), or a graphics processing unit (GPU) or various types of functions may be implemented by software and hardware in cooperation. Also, some or all functions of the controller 140 of the above-described radiation therapy device 100 may be implemented by a dedicated LSI circuit. The program (software) may be stored in the storage 130 or may be stored in a removable storage medium (a non-transitory storage medium) such as a DVD or a CD-ROM and installed in the storage 130 when the storage medium is mounted in a drive device of the radiation therapy system 1. Also, the program (software) may be downloaded in advance from another computer device via the network and installed in the storage 130.

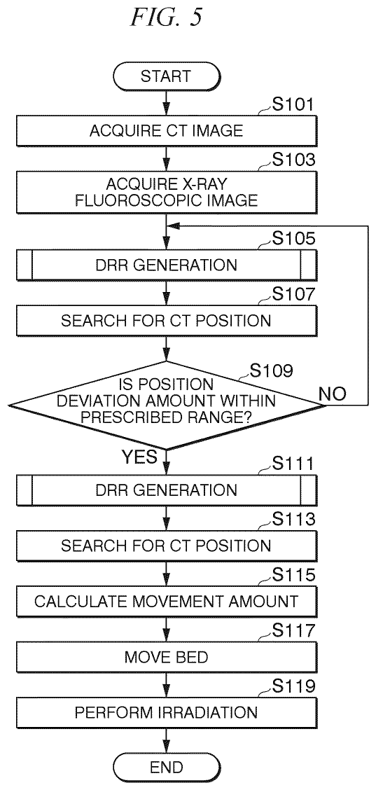

Next, a process of the radiation therapy system 1 will be described. FIG. 5 is a flowchart showing an example of a flow of the process of the radiation therapy system according to the embodiment. Also, in the following description, it is assumed that the CT image D1 of the patient P imaged by the CT device and the treatment plan information D2 are stored in advance in the storage 130 in the treatment planning stage.

First, the first acquirer 151 acquires the CT image D1 of the patient P to be treated from the storage 130 (step S101). The first acquirer 151 outputs the acquired CT data D1 to the DRR generator 155.

Subsequently, the second acquirer 153 acquires a current X-ray fluoroscopic image of the patient P output by the radiation detector 30 (step S103). The second acquirer 153 outputs the acquired X-ray fluoroscopic image to the positioner 157.

Subsequently, the DRR generator 155 and the positioner 157 start a sparse search process for a position of the CT image D1 (hereinafter referred to as a “CT position”) virtually arranged in a three-dimensional space of the treatment room. In the sparse search process for the CT position, the DRR generator 155 generates a DRR on the basis of the CT image D1 output from the first acquirer 151 (step S105). The DRR generator 155 outputs the generated DRR to the positioner 157. Details of the DRR generation process of the DRR generator 155 will be described below.

Subsequently, the positioner 157 searches for a CT position having a highest degree of similarity between the current DRR and the X-ray fluoroscopic image on the basis of the DRR output by the DRR generator 155 and the X-ray fluoroscopic image output by the second acquirer 153 (step S107).

Subsequently, the positioner 157 determines whether or not a position deviation amount of the patient P at the CT position found in the search is within a prescribed range (step S109). The position deviation amount is a position deviation amount between the CT position of the CT image D1 (the position of the patient P in the CT image D1) and the current position of the patient P fixed on the treatment bed 10.

If it is determined that the position deviation amount of the patient P at the CT position found in the search is not within the prescribed range, the positioner 157 outputs information about the CT position found in the search to the DRR generator 155 and returns the process to step S105. Thereby, the DRR generator 155 generates a new DRR on the basis of the information about the CT position output by the positioner 157 in step S105 and the positioner 157 searches for a CT position having a highest degree of similarity between the new DRR and the X-ray fluoroscopic image on the basis of the DRR generated by the DRR generator 155 and the X-ray fluoroscopic image in step S107. In this way, the DRR generator 155 and the positioner 157 cooperate with each other and iterate the sparse search process for the CT position until the position deviation amount of the patient P at the CT position found in the search is within the prescribed range, i.e., until the degree of similarity between the DRR and the X-ray fluoroscopic image is higher than a prescribed threshold value for the degree of similarity.

On the other hand, when it is determined in step S109 that the position deviation amount of the patient P at the CT position found in the search is within the prescribed range, the DRR generator 155 and the positioner 157 start a fine search process for searching for a CT position where the position deviation amount of the patient P is smallest in more detail. In the fine search process for the CT position, the DRR generator 155 generates a DRR based on the CT position found in the sparse search process (step S111). The DRR generator 155 outputs the generated DRR to the positioner 157.

Subsequently, the positioner 157 uses the CT position found in the sparse search process as a reference and searches for a final CT position on the basis of the DRR output by the DRR generator 155 and the X-ray fluoroscopic image output by the second acquirer 153 (step S113). For example, the positioner 157 searches for the CT position where the position deviation amount of the patient P is smallest while moving the CT position along the rotation and translation directions based on the three-dimensional coordinates within the treatment room on the basis of the DRR and the X-ray fluoroscopic image using the CT position found in the sparse search process as the reference. In other words, the positioner 157 moves the CT position according to six parameters representing an amount of rotation and an amount of translation based on the three-dimensional coordinates in the treatment room and searches for a CT position where a degree of similarity between the DRR and the X-ray fluoroscopic image is highest.

Subsequently, the positioner 157 calculates an amount of movement (six control parameters) for rotating and translating the treatment bed 10 on the basis of three-dimensional coordinates in the treatment room on the basis of the final CT position found in the search (step S115). The positioner 157 outputs the calculated amount of movement to the bed controller 159.

Subsequently, the bed controller 159 moves the treatment bed 10 in accordance with the amount of movement output by the positioner 157 (step S117). Subsequently, the irradiation controller 161 controls the treatment beam irradiation gate 40 to irradiate the diseased part of the patient P with the treatment beam B. With the above, the process of the present flowchart ends.

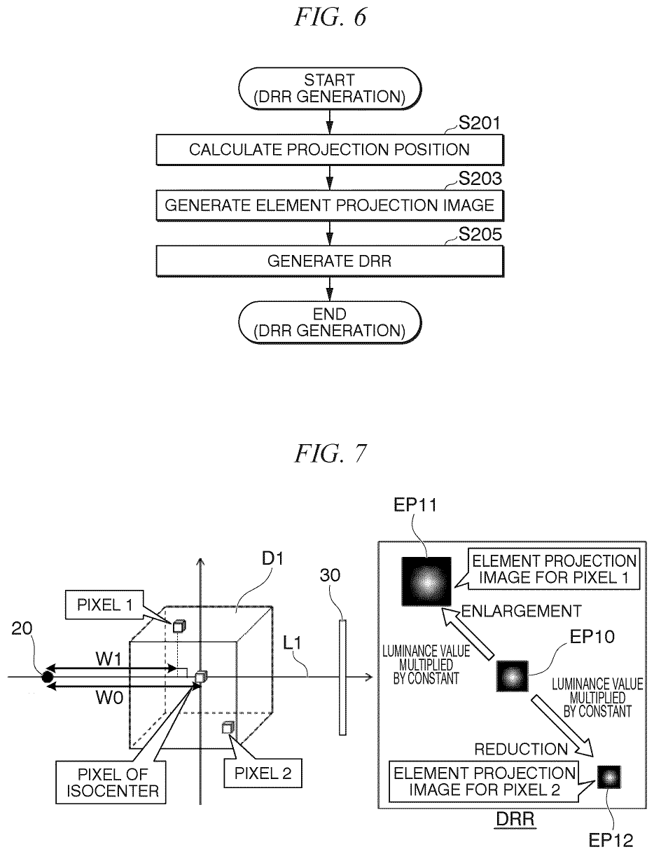

Next, details of the DRR generation process in steps 105 and 111 described above will be described. FIG. 6 is a flowchart showing an example of a flow of the DRR generation process of the DRR generator 155 according to the embodiment.

First, the projection position calculator 201 calculates a projection position when each pixel of the CT image D1 is projected onto the DRR on the basis of the imaging system geometry information D3 (step S201). The projection position calculator 201 converts an image coordinate system set for the CT image D1 into a room coordinate system and multiplies the room coordinate system by a projection matrix based on the imaging system geometry information D3 to calculate a DRR coordinate system at a position on the DRR.

Subsequently, the element projection image generator 203 generates an element projection image when each pixel of the CT image D1 is projected onto the DRR (step S203). Even if the pixels included in the CT image D1 have the same shape, when the position in the three-dimensional space changes, the element projection image also changes. If strictly calculated, it is necessary to generate accurate element projection images for all pixels included in the CT image D1, but the calculation cost is high and it is difficult to generate the DRR at a high speed. Thus, the element projection image generator 203 first generates an element projection image corresponding to a reference pixel and two-dimensionally converts the generated element projection image to approximate an element projection image of a pixel other than the reference pixel.

Subsequently, the element projection image synthesizer 205 generates a DRR by attaching a plurality of element projection images generated by the element projection image generator 203 to projection positions and synthesizing the element projection images (step S205).

FIG. 7 is a diagram showing a state in which an element projection image is generated by the DRR generator 155 according to the embodiment. The DRR generator 155 generates a DRR by generating an element projection image corresponding to a reference pixel, two-dimensionally converting the generated element projection image to generate an element projection image of another pixel, attaching a plurality of generated element projection images to projection positions, and synthesizing the element projection images.

Specifically, an image generated by projecting one pixel of the CT image D1 at a position X(→)=(X, Y, Z)^{t} in the three-dimensional space onto the DRR plane (the radiation detector 30) is set as an element projection image e(u, v). A central position of e(u, v) on the DRR has coordinates eₑ(→)=(eᵤ, eᵥ)^{t} obtained by projecting X(→) onto the DRR. In X(→) and eₑ(→), a relationship of the following Eq. (6) is valid.

$$\begin{matrix}[ Math . 6 ] & \\ λ [ \begin{matrix}{e}_{c} \\ 1\end{matrix} ] = P [ \begin{matrix}X \\ 1\end{matrix} ] & ( 6 )\end{matrix}$$

In the above Eq. (6), P denotes a projection matrix calculated from the imaging system geometry D3. I(u, v) generated by superposing e(u, v) is considered. Because the pixels of the CT image D1 are three-dimensionally arranged in a three-dimensional space and a size of the projected element image is almost larger than a size of a pixel, there are a plurality of element projection images overlapping at coordinates (u, v) on the DRR. Thus, I(u, v) is calculated according to the following Eq. (7).

$$\begin{matrix}[ Math . 7 ] & \\ I ( u , v ) = {\sum }_{e ∈ {E}_{uv}} {e}_{i} ( \frac{{s}_{u}}{{s}_{eu}} ( u - {e}_{{u}_{i}} ) + \frac{{w}_{i} - 1}{2} , \frac{{s}_{v}}{{s}_{ev}} ( v - {e}_{{v}_{i}} ) + \frac{{h}_{i} - 1}{2} ) & ( 7 )\end{matrix}$$

In the above Eq. (7), Eᵤᵥ is a set of element projection images overlapping the coordinates (u, v), wᵢ and hᵢ denote image sizes of i^{th} element projection images in Eᵤᵥ, and sₑᵤ [mm/pixel] and sₑᵥ [mm/pixel] are pixel pitches of the element projection images.

Next, a method of generating e(u, v) will be described. Strictly speaking, it is necessary to generate element projection images for all pixels of the CT image D1, but this requires the same amount of calculation as a process of generating a DRR in the conventional ray tracing method. Thus, the DRR generator 155 simplifies a process by two-dimensionally converting an element projection image corresponding to a reference pixel and approximating an element projection image of another pixel. The reference pixel is, for example, an isocenter pixel that is a site to which radiation is concentrically radiated. Hereinafter, a case where the reference pixel is the isocenter pixel will be described as an example.

A luminance value of the element projection image depends on (is proportional to) a luminance value V(X, Y, Z) of the CT image D1 that is the basis. Thus, the luminance value of the element projection image of the other pixel can be obtained by multiplying a luminance value of the element projection image of the isocenter pixel by a constant. That is, the element projection image generator 203 calculates a ratio of the luminance value of the other pixel to the luminance value of the isocenter pixel in the CT image D1. Also, the element projection image generator 203 can calculate the luminance value of the element projection image of the other pixel by multiplying the luminance value of the element projection image of the isocenter pixel by the calculated ratio.

Also, the element projection image of the pixel included in the CT image D1 becomes larger when it is closer to the radiation source 20 and becomes smaller when it is closer to the radiation detector 30 (farther away from the radiation source 20). In other words, the element projection image becomes larger if a position of the other pixel is closer to the radiation source 20 than a position of the isocenter pixel and becomes smaller if the position of the other pixel is closer to the radiation detector 30 (DRR) than the position of the isocenter pixel. The size of the element projection image considering such a tendency can be calculated geometrically. Thus, the element projection image generator 203 can perform a conversion process in consideration of a difference in the position of each pixel included in the CT image D1 by enlarging or reducing the size of the element projection image.

An element projection image generated in the ray tracing method for the pixel at the isocenter position (Xᵢₛₒ, Yᵢₛₒ, Zᵢₛₒ) is set as a reference element projection image eᵢₛₒ(u, v). If a result of approximating the element projection image of a pixel at a position (Xᵢ, Yᵢ, Zᵢ) other than the isocenter in a two-dimensional conversion process for eᵢₛₒ(u, v) is set as eᵢ(u, v), a conversion equation is represented according to the following Eqs. (8), (9), and (10).

$$\begin{matrix}[ Math . 8 ] & \\ {e}_{i} ( u , v ) = α {e}_{iso} ( β ( u - \frac{{w}_{i} - 1}{2} ) + \frac{{w}_{iso} - 1}{2} , β ( v - \frac{{h}_{i} - 1}{2} ) + \frac{{h}_{iso} - 1}{2} ) & ( 8 )\end{matrix}$$

$$\begin{matrix}[ Math . 9 ] & \\ α = \frac{V ( {X}_{i} , {Y}_{i} , {Z}_{i} )}{V ( {X}_{iso} , {Y}_{iso} , {Z}_{iso} )} & ( 9 )\end{matrix}$$

$$\begin{matrix}[ Math . 10 ] & \\ β = \frac{{λ}_{i}}{{λ}_{iso}} & ( 10 )\end{matrix}$$

In the above Eqs. (8) to (10), w and h denote sizes of the element projection images. α is a ratio of the pixel values of the CT image D1 serving as the basis of each element projection image. Because the pixel value of the CT image D1 is not related to the pixel position and it is difficult to approximate a luminance value of an element projection image of another pixel of a processing target (hereinafter also referred to as a “pixel of interest”) only by resizing the reference element projection image, a ratio of the pixel values is corrected. λᵢ and λᵢₛₒ are calculated according to the above Eq. (6) and β denotes a ratio between the depth from the radiation source 20 to the pixel of interest and the depth from the radiation source 20 to the isocenter position. The depth from the radiation source 20 to the pixel of interest is, for example, a distance W1 between a point obtained by dropping a perpendicular line from the position of the pixel of interest (for example, pixel 1) to a straight line L1 connecting the radiation source 20 and the position of the isocenter and the radiation source 20 (see FIG. 7). The depth from the radiation source 20 to the isocenter position is, for example, a linear distance W0 from the radiation source 20 to the isocenter position (see FIG. 7).

In the example shown in FIG. 7, first, the element projection image generator 203 projects a pixel at the isocenter among the pixels included in the CT image D1 to generate a reference element projection image EP10. Subsequently, the element projection image generator 203 generates an element projection image EP11 of pixel 1, which is another pixel among the pixels included in the CT image D1. Here, pixel 1 is located closer to the radiation source 20 than the isocenter. Thus, the element projection image generator 203 generates the element projection image EP11 (an enlarged element projection image) larger in size than the reference element projection image EP10 on the basis of the above-described depth ratio. Also, the element projection image generator 203 calculates a luminance value of the element projection image EP11 by multiplying the reference element projection image EP10 by a ratio of the luminance value of pixel 1 to the luminance value of the pixel of the isocenter position.

Likewise, the element projection image generator 203 generates an element projection image EP12 of pixel 2, which is another pixel, among the pixels included in the CT image D1. Here, pixel 2 is located closer to the radiation detector 30 than the isocenter. Thus, the element projection image generator 203 generates the element projection image EP12 by reducing the size of the reference element projection image EP10 on the basis of the above-described depth ratio. Also, the element projection image generator 203 calculates a luminance value of the element projection image EP12 by multiplying the reference element projection image EP10 by a ratio of a luminance value of pixel 2 to the luminance value of the pixel at the isocenter position.

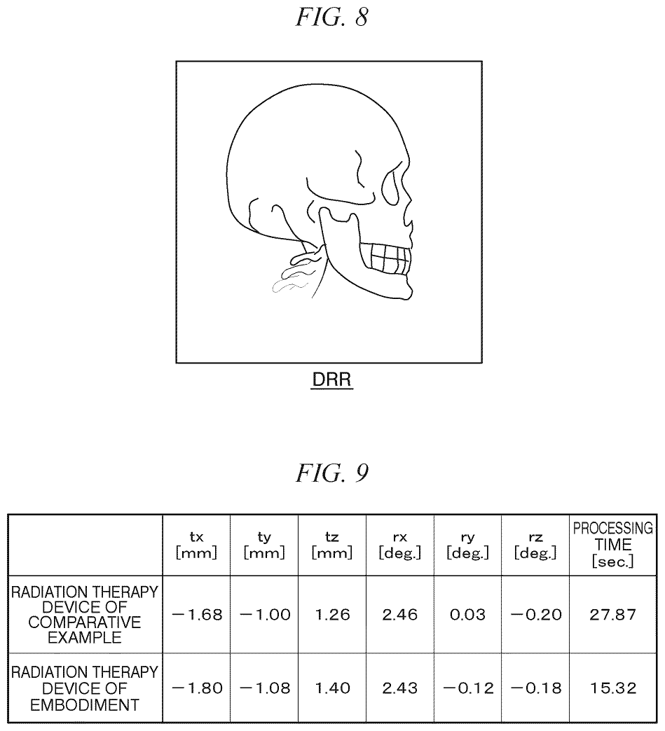

The element projection image generator 203 similarly generates element projection images for the remaining pixels included in the CT image D1. The element projection image synthesizer 205 can generate a DRR as shown in FIG. 8 by attaching a plurality of element projection images generated by the element projection image generator 203 to projection positions and synthesizing the element projection images.

That is, the element projection image generator 203 virtually arranges the three-dimensional image between a radiation source for performing the X-ray imaging and a radiation detector, generates the element projection image of the other pixel by performing a conversion process of enlarging the element projection image of the reference pixel when the other pixel is closer to the radiation source than the reference pixel, and generates the element projection image of the other pixel by performing a conversion process of reducing the element projection image of the reference pixel when the other pixel is closer to the radiation detector than the reference pixel. Also, the element projection image generator 203 calculates a luminance value of the element projection image of the other pixel on the basis of a ratio between a luminance value of the reference pixel included in the three-dimensional image and a luminance value of the other pixel. The element projection image generator 203 calculates the luminance value of the element projection image of the other pixel by multiplying the luminance value of the element projection image of the reference pixel by a ratio of the luminance value of the other pixel to a luminance value of the reference pixel.

As described above, because exact conversion is not possible in a two-dimensional conversion process, an approximation error of the element projection image increases as a distance of a pixel from the isocenter in a direction horizontal to the radiation detector 30 increases. In patient positioning, the diseased part is located at the isocenter as a desired location in a positioning process with highest accuracy. That is, if the DRR is generated on the basis of the element projection image generated by the pixel at the isocenter position as in the present embodiment, it is possible to suppress an error near the isocenter.

FIG. 9 is a diagram showing experimental results of positioning processes of the radiation therapy device 100 according to the embodiment and a device of a comparative example. In this experiment, in each of a case where a DRR is generated using a computer having specific processing performance and using an element projection image according to the embodiment and a case where a DRR is generated in a conventional ray casting method of the comparative example, an amount of movement was calculated by performing a positioning process (a sparse search or a fine search) from an appropriate initial position. In FIG. 9, tx, ty, and tz denote amounts of movement in three axial directions in the translation mechanism and rx, ry, and rz denote amounts of movement in three axial directions in the rotation mechanism. As shown in FIG. 9, it was found that the processing time in the case of a process using the element projection images according to the embodiment could be significantly reduced compared to the processing time in the case where the conventional ray casting method of the comparative example was adopted.

According to the above-described embodiment, by generating and synthesizing element projection images from three-dimensional images, a DRR generation process can be speeded up and the patient can be positioned in a short time with high accuracy.

Also, when the CT image D1 is a three-dimensional rectangular parallelepiped image, the DRR generator 155 may be configured to perform an isotropic process for the CT image D1 (a process of converting the CT image D1 into a cube) and then perform a projection image generation process. Because a cube is more likely to be a nearby element projection image than a rectangular parallelepiped even if the cube is projected from any angle, variations in the element projection images can be suppressed.

Also, the DRR generator 155 may obtain a projection image of one pixel by setting one pixel having a luminance of 1 at a reference position (for example, the isocenter position). Thereby, it is possible to reduce an error in the image near the isocenter at which a highly accurate DRR is necessary. In this case, the luminance value can be represented by a constant multiple and the movement in the depth direction can be represented by changing the scale. Also, a pixel size of the CT image D1 may be reduced. Thereby, a DRR with high image quality can be generated.

According to at least one embodiment described above, there are provided an acquirer (151) configured to acquire a condition of X-ray imaging in a treatment stage and a three-dimensional image of a patient imaged before the treatment stage; a projection position calculator (201) configured to calculate a projection position when each of pixels included in the three-dimensional image is projected onto a two-dimensional X-ray fluoroscopic image generated in the X-ray imaging on the basis of the condition of the X-ray imaging; an element projection image generator (203) configured to generate an element projection image for each pixel when each of the pixels included in the three-dimensional image has been projected onto the X-ray fluoroscopic image; and an element projection image synthesizer (205) configured to perform a synthesis process for the generated element projection image for each pixel on the basis of the calculated projection position to generate a reconstructed image (DDR) obtained by virtually reproducing the X-ray fluoroscopic image from the three-dimensional image, whereby a DRR generation process can be speeded up and the patient can be positioned in a short time with high accuracy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A radiation therapy device comprising:
an acquirer configured to acquire a condition of X-ray imaging in a treatment stage and a three-dimensional image of a patient imaged before the treatment stage;
a projection position calculator configured to calculate a projection position when each of pixels included in the three-dimensional image is projected onto a two-dimensional X-ray fluoroscopic image generated in the X-ray imaging on the basis of the condition of the X-ray imaging;
an element projection image generator configured to generate an element projection image for each pixel when each of the pixels included in the three-dimensional image is projected onto the X-ray fluoroscopic image; and
an element projection image synthesizer configured to perform a synthesis process for the generated element projection image for each pixel on the basis of the calculated projection position to generate a reconstructed image virtually reproducing the X-ray fluoroscopic image from the three-dimensional image.

2. The radiation therapy device according to claim 1, further comprising a positioner configured to perform a positioning process for the patient on the basis of the generated reconstructed image.

3. The radiation therapy device according to claim 1,
wherein the element projection image generator is configured to
generate an element projection image of a reference pixel included in the three-dimensional image, and
generate an element projection image of a pixel other than the reference pixel included in the three-dimensional image by performing a two-dimensional conversion process for the generated element projection image of the reference pixel.

4. The radiation therapy device according to claim 3, wherein the reference pixel is a pixel of a position of an isocenter in radiation therapy.

5. The radiation therapy device according to claim 3,
wherein the element projection image generator is configured to
virtually arrange the three-dimensional image between a radiation source for performing the X-ray imaging and a radiation detector,
generate the element projection image of the other pixel by performing a conversion process of enlarging the element projection image of the reference pixel when the other pixel is closer to the radiation source than the reference pixel, and
generate the element projection image of the other pixel by performing a conversion process of reducing the element projection image of the reference pixel when the other pixel is closer to the radiation detector than the reference pixel.

6. The radiation therapy device according to claim 3, wherein the element projection image generator is configured to calculate a luminance value of the element projection image of the other pixel on the basis of a ratio between a luminance value of the reference pixel included in the three-dimensional image and a luminance value of the other pixel.

7. The radiation therapy device according to claim 6, wherein the element projection image generator is configured to calculate the luminance value of the element projection image of the other pixel by multiplying the luminance value of the element projection image of the reference pixel by a ratio of the luminance value of the other pixel to a luminance value of the reference pixel.

8. A medical image processing device comprising:
an acquirer configured to acquire a condition of X-ray imaging in a treatment stage and a three-dimensional image of a patient imaged before the treatment stage;
a projection position calculator configured to calculate a projection position when each of pixels included in the three-dimensional image is projected onto a two-dimensional X-ray fluoroscopic image generated in the X-ray imaging on the basis of the condition of the X-ray imaging;
an element projection image generator configured to generate an element projection image for each pixel when each of the pixels included in the three-dimensional image is projected onto the X-ray fluoroscopic image; and
an element projection image synthesizer configured to perform a synthesis process for the generated element projection image for each pixel on the basis of the calculated projection position to generate a reconstructed image virtually reproducing the X-ray fluoroscopic image from the three-dimensional image.

9. A radiation therapy method comprising:
acquiring, by a computer, a condition of X-ray imaging in a treatment stage and a three-dimensional image of a patient imaged before the treatment stage;
calculating, by the computer, a projection position when each of pixels included in the three-dimensional image is projected onto a two-dimensional X-ray fluoroscopic image generated in the X-ray imaging on the basis of the condition of the X-ray imaging;
generating, by the computer, an element projection image for each pixel when each of the pixels included in the three-dimensional image is projected onto the X-ray fluoroscopic image; and
performing, by the computer, a synthesis process for the generated element projection image for each pixel on the basis of the calculated projection position to generate a reconstructed image virtually reproducing the X-ray fluoroscopic image from the three-dimensional image.

10. A non-transitory computer-readable storage medium storing a program for causing a computer to:
acquire a condition of X-ray imaging in a treatment stage and a three-dimensional image of a patient imaged before the treatment stage;
calculate a projection position when each of pixels included in the three-dimensional image is projected onto a two-dimensional X-ray fluoroscopic image generated in the X-ray imaging on the basis of the condition of the X-ray imaging;
generate an element projection image for each pixel when each of the pixels included in the three-dimensional image is projected onto the X-ray fluoroscopic image; and
perform a synthesis process for the generated element projection image for each pixel on the basis of the calculated projection position to generate a reconstructed image virtually reproducing the X-ray fluoroscopic image from the three-dimensional image.
